(19) United States
(12) Patent Application Publication
(10) Pub. No.: US 2024/0122990 A1
(43) Pub. Date: Apr. 18, 2024

(54) COMPOSITIONS AND METHODS FOR CARDIOMYOCYTE TRANSPLANTATION

(71) Applicant: UNIVERSITY OF WASHINGTON, Seattle, WA (US)

(72) Inventors: Charles E. MURRY, Seattle, WA (US); William Robb MACLELLAN, Seattle, WA (US); Robert Scott THIES, Seattle, WA (US); Kenta NAKAMURA, Seattle, WA (US); Daisy Sue NAKAMURA, Seattle, WA (US); Lauren E. NEIDIG, Seattle, WA (US)

(73) Assignee: UNIVERSITY OF WASHINGTON, Seattle, WA (US)

(21) Appl. No.: 18/278,085

(22) PCT Filed: Feb. 22, 2022
(86) PCT No.: PCT/US2022/017268

§ 371 Date: Aug. 21, 2023

Related U.S. Application Data
(60) Provisional application No. 63/152,737, filed on Feb. 23, 2021.

(51) Int. Cl. A61K 35/34; A61K 31/436; A61K 38/17; A61P 37/06

(52) U.S. Cl. CPC A61K 35/34; A61K 31/436; A61K 38/1774; A61P 37/06

(57) ABSTRACT

The present disclosure provides for the treatment of cardiac diseases and disorders using in vitro-differentiated cardiomyocytes. Such methods can take advantage of both autologous and allogeneic pluripotent stem cells.

### CROSS-REFERENCE TO RELATED APPLICATIONS

This application claims benefit under 35 U.S.C. § 119(e) of U.S. Provisional Application No. 63/152,737 filed Feb. 23, 2021, the contents of which are incorporated herein by reference in their entirety.

### STATEMENT OF FEDERALLY SPONSORED RESEARCH AND DEVELOPMENT

This invention was made with government support under Grant No. R01 HL128362 awarded by the National Institutes of Health. The government has certain rights in the invention.

### BACKGROUND

The field of regenerative medicine for the treatment of cardiac diseases and disorders is advancing and the use of in vitro-differentiated cardiomyocytes is being explored in the clinical setting. Such methods can take advantage of both autologous and allogeneic pluripotent stem cells. In an effort to minimize rejection of cardiac grafts following transplantation, there is a need to identify therapies that can be used to prevent short-term rejection and promote maintenance of a cardiac graft for long periods of time.

### SUMMARY

The methods and compositions described herein are based, in part, on the discovery of a combination of a calcineurin inhibitor and an inhibitor of CD80/CD86 signaling that can prevent or reduce rejection of a graft of in vitro-differentiated cardiomyocytes in a subject being treated for a cardiac disease or disorder.

Accordingly, provided herein in one aspect is a method of cardiomyocyte transplantation, the method comprising: (i) administering in vitro-differentiated cardiomyocytes or cardiac progenitor cells to a graft site in cardiac tissue of a recipient in need thereof; and (ii) administering a calcineurin inhibitor and an inhibitor of CD80 and/or CD86 signaling to the recipient.

In one embodiment of this aspect and all other aspects provided herein, the in vitro-differentiated cardiomyocytes are allogeneic to the recipient.

In another embodiment of this aspect and all other aspects provided herein, the in vitro-differentiated cardiomyocytes are autologous to the recipient.

In another embodiment of this aspect and all other aspects provided herein, the calcineurin inhibitor is selected from the group consisting of: tacrolimus, cyclosporine, voclosporin, and pimecrolimus. Also contemplated herein are immunosuppressive derivatives of tacrolimus, cyclosporine, voclosporin and pimecrolimus.

In another embodiment of this aspect and all other aspects provided herein, the inhibitor of CD80 and/or CD86 comprises an extracellular domain fragment of CTLA4.

In another embodiment of this aspect and all other aspects provided herein, the extracellular domain fragment of CTLA4 is fused to a heterologous polypeptide.

In another embodiment of this aspect and all other aspects provided herein, the heterologous polypeptide increases the serum half-life of the inhibitor of CD80 and/or CD86.

In another embodiment of this aspect and all other aspects provided herein, the heterologous polypeptide comprises an IgG Fc domain polypeptide.

In another embodiment of this aspect and all other aspects provided herein, the extracellular domain fragment of CTLA4 is conjugated with polyethylene glycol (PEG).

In another embodiment of this aspect and all other aspects provided herein, the inhibitor of CD80 and/or CD86 signaling comprises abatacept or belatacept.

In another embodiment of this aspect and all other aspects provided herein, abatacept is administered at a dose between 5 mg/kg and 20 mg/kg via intravenous or subcutaneous administration. In another embodiment of this aspect and all other aspects provided herein, abatacept is administered at a dose between 10 mg/kg and 15 mg/kg via intravenous or subcutaneous administration (e.g., at a dose of 12.5 mg/kg).

In another embodiment of this aspect and all other aspects provided herein, the inhibitor of CD80 and/or CD86 signaling is (i) administered every 2 weeks or every 3 weeks, (ii) administered every 12-16 days, (iii) administered every 10-18 days, and/or (iv) administered by subcutaneous, oral or intravenous administration. That is, the inhibitor of CD80 and/or CD86 signaling can be administered every 10, every 11, every 12, every 13, every 14, every 15, every 16, every 17, every 18, every 19, every 20, every 21, every 22, every 23, every 24 days or more.

In another embodiment of this aspect and all other aspects provided herein, the calcineurin inhibitor comprises tacrolimus or an immunosuppressive derivative thereof.

In another embodiment of this aspect and all other aspects provided herein, tacrolimus is administered to achieve a serum or plasma concentration of 5-20 ng/ml. In other embodiments, tacrolimus is administered to achieve a blood, plasma or serum concentration between 10 mg/kg and 15 mg/kg via intravenous or subcutaneous administration.

In another embodiment of this aspect and all other aspects provided herein, the tacrolimus is administered via a continuous infusion, every 2 hours, every 4 hours, every 6 hours, every 8 hours, every 10 hours, every 12 hours, ever 14 hours, every 16 hours, every 18 hours, every 20 hours, every 22 hours, or every 24 hours (i.e., daily).

In another embodiment of this aspect and all other aspects provided herein, the calcineurin inhibitor and the inhibitor of CD80 and/or CD86 signaling are each administered subcutaneously or intravenously. Alternatively, the calcineurin inhibitor can be administered subcutaneously while the inhibitor of CD80 and/or CD86 is administered intravenously, or vice versa.

In another embodiment of this aspect and all other aspects provided herein, immune rejection of the administered cardiomyocytes is reduced (e.g., by at least 10%) relative to rejection of cardiomyocytes administered without the calcineurin inhibitor and the inhibitor of CD80 and/or CD86 signaling.

In another embodiment of this aspect and all other aspects provided herein, the need for steroid immunosuppression to suppress immune rejection of the transplanted cardiomyocytes is reduced relative to the need for steroid immunosuppression when cardiomyocytes are transplanted without administering the calcineurin inhibitor and the inhibitor of CD80 and/or CD86 signaling.

In another embodiment of this aspect and all other aspects provided herein, CD3+ T cell or CD20+ B cell infiltration into the graft site is reduced relative to infiltration occurring in the absence of administering the calcineurin inhibitor and the inhibitor of CD80 and/or CD86 signaling.

In another embodiment of this aspect and all other aspects provided herein, CD3+ T cell or CD20+ B cell infiltration into the graft site is reduced at 8 weeks post-administration of the cardiomyocytes.

In another embodiment of this aspect and all other aspects provided herein, CD3+ T cell or CD20+ B cell infiltration into the graft site remains reduced at 16 weeks post-administration of the cardiomyocytes.

Another aspect provided herein relates to a method of reducing immune rejection of transplanted, in vitro-differentiated cardiomyocytes in a recipient, the method comprising administering a calcineurin inhibitor and an inhibitor of CD80 and/or CD86 signaling to the recipient.

In one embodiment of this aspect and all other aspects provided herein, the in vitro-differentiated cardiomyocytes are allogeneic to the recipient.

In another embodiment of this aspect and all other aspects provided herein, the in vitro-differentiated cardiomyocytes are autologous to the recipient.

In another embodiment of this aspect and all other aspects provided herein, the calcineurin inhibitor is selected from the group consisting of: tacrolimus, cyclosporine, voclosporin, and pimecrolimus.

In another embodiment of this aspect and all other aspects provided herein, the inhibitor of CD80 and/or CD86 comprises an extracellular domain fragment of CTLA4.

In another embodiment of this aspect and all other aspects provided herein, the extracellular domain fragment of CTLA4 is fused to a heterologous polypeptide.

In another embodiment of this aspect and all other aspects provided herein, the heterologous polypeptide increases the serum half-life of the inhibitor of CD80 and/or CD86.

In another embodiment of this aspect and all other aspects provided herein, the heterologous polypeptide comprises an IgG Fc domain polypeptide.

In another embodiment of this aspect and all other aspects provided herein, the extracellular domain fragment of CTLA4 is conjugated with polyethylene glycol (PEG).

In another embodiment of this aspect and all other aspects provided herein, the inhibitor of CD80 and/or CD86 signaling comprises abatacept or belatacept.

In another embodiment of this aspect and all other aspects provided herein, abatacept is administered at a dose between 1.25 mg/kg and 125 mg/kg via intravenous or subcutaneous administration.

In another embodiment of this aspect and all other aspects provided herein, the inhibitor of CD80 and/or CD86 signaling is (i) administered every 2 weeks or every 3 weeks, (ii) administered every 12-16 days, (iii) administered every 10-18 days, and/or (iv) administered by subcutaneous, oral or intravenous administration. That is, the inhibitor of CD80 and/or CD86 signaling can be administered every 10, every 11, every 12, every 13, every 14, every 15, every 16, every 17, every 18, every 19, every 20, every 21, every 22, every 23, every 24 days or more.

In another embodiment of this aspect and all other aspects provided herein, the calcineurin inhibitor comprises tacrolimus or an immunosuppressive derivative thereof.

In another embodiment of this aspect and all other aspects provided herein, wherein tacrolimus is administered to achieve a serum or plasma concentration of 5-20 ng/ml.

In another embodiment of this aspect and all other aspects provided herein, the tacrolimus is administered via a continuous infusion, every 2 hours, every 4 hours, every 6 hours, every 8 hours, every 10 hours, every 12 hours, ever 14 hours, every 16 hours, every 18 hours, every 20 hours, every 22 hours, or daily.

In another embodiment of this aspect and all other aspects provided herein, the calcineurin inhibitor and the inhibitor of CD80 and/or CD86 signaling are each administered subcutaneously or intravenously or a combination thereof.

In another embodiment of this aspect and all other aspects provided herein, immune rejection of the administered cardiomyocytes is reduced relative to rejection of cardiomyocytes administered without the calcineurin inhibitor and the inhibitor of CD80 and/or CD86 signaling.

In another embodiment of this aspect and all other aspects provided herein, the need for steroid immunosuppression to suppress immune rejection of the transplanted cardiomyocytes is reduced relative to the need for steroid immunosuppression when cardiomyocytes are transplanted without administering the calcineurin inhibitor and the inhibitor of CD80 and/or CD86 signaling.

In another embodiment of this aspect and all other aspects provided herein, CD3+ T cell or CD20+ B cell infiltration into the graft site is reduced relative to infiltration occurring in the absence of administering the calcineurin inhibitor and the inhibitor of CD80 and/or CD86 signaling.

In another embodiment of this aspect and all other aspects provided herein, CD3+ T cell or CD20+ B cell infiltration into the graft site is reduced at 8 weeks post-administration of the cardiomyocytes.

In another embodiment of this aspect and all other aspects provided herein, CD3+ T cell or CD20+ B cell infiltration into the graft site remains reduced at 16 weeks post-administration of the cardiomyocytes.

Another aspect provided herein is a method of treating a cardiovascular disease or disorder, the method comprising: (i) administering in vitro-differentiated cardiomyocytes to a graft site in cardiac tissue of a subject in need thereof, and (ii) administering a calcineurin inhibitor and an inhibitor of CD80 and/or CD86 signaling to the subject.

In one embodiment of this aspect and all other aspects provided herein, the cardiovascular disease or disorder comprises myocardial infarction, ischemia/reperfusion injury, tachyarrhythmias, bradyarrhhythmias, cardiomyopathy, congenital defects, and muscular dystrophy-associated cardiomyopathy.

In another embodiment of this aspect and all other aspects provided herein, the congenital defects comprise septal defects or hypoplastic syndromes.

In another embodiment of this aspect and all other aspects provided herein, the muscular-dystrophy associated cardiomyopathy comprises cardiomyopathy associated with Duchenne's muscular dystrophy (DMD).

In another embodiment of this aspect and all other aspects provided herein, the in vitro-differentiated cardiomyocytes are allogeneic to the recipient.

In another embodiment of this aspect and all other aspects provided herein, the in vitro-differentiated cardiomyocytes are autologous to the recipient.

In another embodiment of this aspect and all other aspects provided herein, the calcineurin inhibitor is selected from the group consisting of: tacrolimus, cyclosporine, voclosporin, and pimecrolimus.

In another embodiment of this aspect and all other aspects provided herein, the inhibitor of CD80 and/or CD86 comprises an extracellular domain fragment of CTLA4.

In another embodiment of this aspect and all other aspects provided herein, the extracellular domain fragment of CTLA4 is fused to a heterologous polypeptide.

In another embodiment of this aspect and all other aspects provided herein, the heterologous polypeptide increases the serum half-life of the inhibitor of CD80 and/or CD86.

In another embodiment of this aspect and all other aspects provided herein, the heterologous polypeptide comprises an IgG Fc domain polypeptide.

In another embodiment of this aspect and all other aspects provided herein, the extracellular domain fragment of CTLA4 is conjugated with polyethylene glycol (PEG).

In another embodiment of this aspect and all other aspects provided herein, the inhibitor of CD80 and/or CD86 signaling comprises abatacept or belatacept.

In another embodiment of this aspect and all other aspects provided herein, abatacept is administered at a dose between 5 mg/kg and 20 mg/kg via intravenous or subcutaneous administration.

In another embodiment of this aspect and all other aspects provided herein, the inhibitor of CD80 and/or CD86 signaling is (i) administered every 2 weeks or every 3 weeks, (ii) administered every 12-16 days, (iii) administered every 10-18 days, and/or (iv) administered by subcutaneous, oral or intravenous administration.

In another embodiment of this aspect and all other aspects provided herein, the calcineurin inhibitor comprises tacrolimus or an immunosuppressive derivative thereof.

In another embodiment of this aspect and all other aspects provided herein, tacrolimus is administered to achieve a serum or plasma concentration of 5-20 ng/ml.

In another embodiment of this aspect and all other aspects provided herein, tacrolimus is administered via a continuous infusion, every 2 hours, every 4 hours, every 6 hours, every 8 hours, every 10 hours, every 12 hours, ever 14 hours, every 16 hours, every 18 hours, every 20 hours, every 22 hours, or daily.

In another embodiment of this aspect and all other aspects provided herein, the calcineurin inhibitor and the inhibitor of CD80 and/or CD86 signaling are each administered subcutaneously or intravenously or a combination thereof.

In another embodiment of this aspect and all other aspects provided herein, immune rejection of the administered cardiomyocytes is reduced relative to rejection of cardiomyocytes administered without the calcineurin inhibitor and the inhibitor of CD80 and/or CD86 signaling.

In another embodiment of this aspect and all other aspects provided herein, the need for steroid immunosuppression to suppress immune rejection of the transplanted cardiomyocytes is reduced relative to the need for steroid immunosuppression when cardiomyocytes are transplanted without administering the calcineurin inhibitor and the inhibitor of CD80 and/or CD86 signaling.

In another embodiment of this aspect and all other aspects provided herein, CD3+ T cell or CD20+ B cell infiltration into the graft site is reduced relative to infiltration occurring in the absence of administering the calcineurin inhibitor and the inhibitor of CD80 and/or CD86 signaling.

In another embodiment of this aspect and all other aspects provided herein, CD3+ T cell or CD20+ B cell infiltration into the graft site is reduced at 8 weeks post-administration of the cardiomyocytes.

In another embodiment of this aspect and all other aspects provided herein, CD3+ T cell or CD20+ B cell infiltration into the graft site remains reduced at 16 weeks post-administration of the cardiomyocytes.

Another aspect provided herein relates to the use of a calcineurin inhibitor in combination with an inhibitor of CD80 and/or CD86 signaling as described herein for the treatment or prevention of immune-mediated rejection of a cardiac graft.

Another aspect provided herein relates to the use of in vitro-differentiated cardiomyocytes or cardiac progenitor cells for cardiomyocyte transplantation, the use comprising: (i) in vitro-differentiated cardiomyocytes or cardiac progenitor cells to be administered to a graft site in cardiac tissue of a recipient in need thereof, and (ii) a calcineurin inhibitor and an inhibitor of CD80 and/or CD86 signaling that is to be administered to the recipient.

Also provided herein, in another aspect, is the use of in vitro-differentiated cardiomyocytes for treating a cardiovascular disease or disorder, the use comprising: (i) in vitro-differentiated cardiomyocytes to a graft site in cardiac tissue to be administered to a subject in need thereof, and (ii) a calcineurin inhibitor and an inhibitor of CD80 and/or CD86 signaling to be administered to the subject.

### BRIEF DESCRIPTION OF THE DRAWINGS

FIG. 1. Histological analysis showing that without immunosuppression, cardiomyocyte grafts survived for 2 weeks without rejection, but by 8 weeks they underwent severe cellular rejection with extensive inflammatory infiltration. Steroid-sparing combination of Tacrolimus+Abatacept revealed low-moderate immune rejection of the graft at 16 weeks with patchy infiltration of CD20+(B-cell) and CD3+(T-cell) populations.

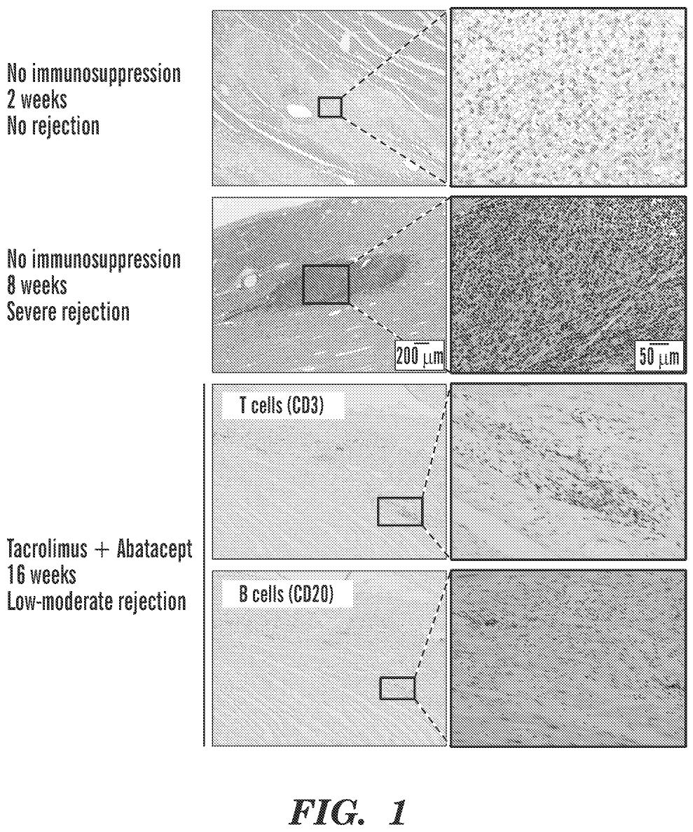

FIG. 2. Schematic showing an exemplary method for in vitro-differentiation of cardiomyocytes from human embryonic stem cells.

### DETAILED DESCRIPTION

Provided herein are methods and compositions comprising combination treatment with a calcineurin inhibitor (e.g., tacrolimus) and an inhibitor of CD80 and/or CD86 signaling (e.g., abatacept) that improve methods of cardiac repair using pluripotent stem cell-derived cardiomyocytes (PSC-CM) by reducing or preventing immune-mediated transplant rejection. The combinatorial immunosuppression regimen of a calcineurin inhibitor (e.g., tacrolimus (TAC)) and an inhibitor of CD80 and/or CD86 signaling (e.g., abatacept (ABT)) can be used to support long-term PSC-CM transplantation.

### Definitions

As used herein, the term “a recipient in need thereof” refers to a subject having a cardiovascular disease or disorder that can be treated with a cardiac graft comprising in vitro-differentiated cardiomyocytes. For example, the recipient in need thereof can comprise a cardiovascular disease or disorder, including an injury, such as: a myocardial infarction, arrhythmias (e.g., tachyarrhythmias, or bradyarrhythmias), ischemia/reperfusion injury, cardiomyopathy, muscular dystrophy-associated cardiomyopathies (e.g., Duchenne's muscular dystrophy (DMD)), or a congenital defect (e.g., hypoplastic syndromes, septal defects) and the like.

The term “differentiate”, or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte progenitor cell), and then to an end-stage differentiated cell (e.g., a cardiomyocyte), which plays a characteristic role in a certain tissue type, and may or may not retain the capacity to proliferate further.

The term “pluripotent” or “pluripotent stem cell (PSC)” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

As used herein, the terms “induced pluripotent stem cell,” “iPSC,” “hPSC,” and “human pluripotent stem cell” are used interchangeably herein and refer to a pluripotent cell artificially derived from a differentiated somatic cell (e.g., by reprogramming using one or more methods known in the art). iPSCs are capable of self-renewal and differentiation into cell fate-committed stem cells, including cells of the cardiac lineages, as well as various types of mature cells.

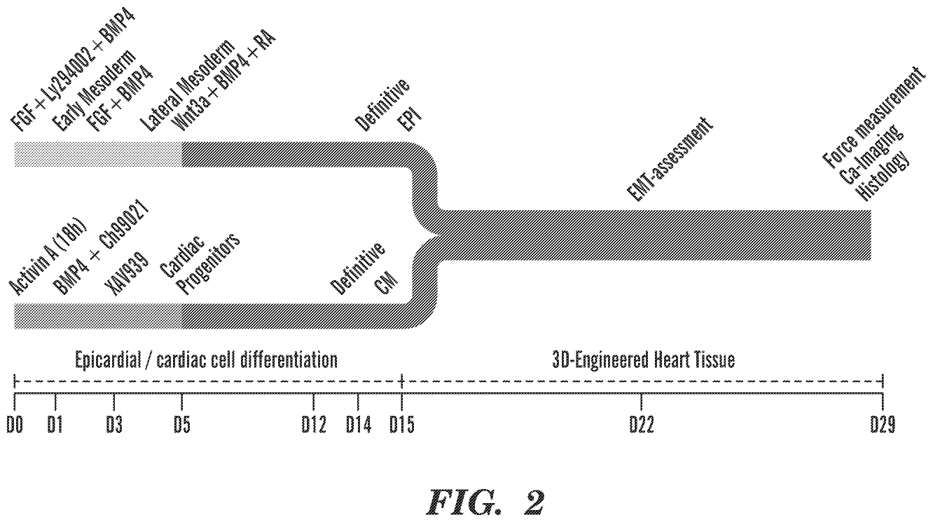

As used herein, “in vitro-differentiated cardiomyocytes” refers to cardiomyocytes that are generated in culture, typically, but not necessarily via step-wise differentiation from a precursor cell such as a human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. As used herein, a cell differentiated in vitro from a stem cell, e.g., an induced pluripotent stem (iPS) cell or embryonic stem cell (“ES cell” or “ESC”), is a “stem-cell derived cardiomyocyte” or “in vitro-differentiated cardiomyocyte” if it has expression of cardiac troponin T (cTnT). Methods for differentiating stem cells in vitro to cardiomyocytes are known in the art and described elsewhere herein. In one embodiment, the cardiomyocytes are differentiated from pluripotent stem cells (e.g., PSC-CMs). An exemplary protocol for in vitro differentiation of cardiomyocytes is depicted in FIG. 2.

As used herein, the term “cardiomyocyte” refers to a cardiac muscle cell. Cardiomyocytes generally comprise phenotypic and/or structural features associated with cardiac muscle (e.g., electrical phenotypes, sarcomeres, actin, myosin and cardiac troponin T expression, etc.). Typically, cardiomyocytes are terminally differentiated.

As used herein, the term “cardiac progenitor cell” refers to a cell that is committed to the cardiac lineage but is not a fully differentiated cardiomyocyte. A cardiac progenitor cell can be differentiated in vivo to a cardiomyocyte within the cardiac graft. In some embodiments, the “cardiac progenitor cell” is a cell that is partially, but not fully, differentiated in vitro along the cardiac lineage. In one embodiment, the cardiac progenitor cell for use with the methods and compositions described herein refers to a partially in vitro-differentiated cardiomyocyte that is paused in the differentiation process following inhibition of the Wnt pathway (see e.g., FIG. 2). In another embodiment, the cardiac progenitor cell comprises a cell at day 5 or later (but prior to terminal differentiation of a cardiomyocyte is reached) of an in vitro-differentiation protocol for generating cardiomyocytes.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes, refers to a population of cells that contains fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

The term “marker” as used herein is used to describe a characteristic and/or phenotype of a cell. Markers can be used, for example, for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell. Similarly, “PSC-CMs” or “pluripotent stem cell-derived cardiomyocytes” are used interchangeably to refer to cardiomyocytes derived from a pluripotent stem cell. In some embodiments, the terms “hPSC-CM” or “human pluripotent stem cell derived cardiomyocytes” are used interchangeably to refer to cardiomyocytes derived from a human pluripotent stem cell.

As used herein, the terms, “maturation” or “mature phenotype” or “mature cardiomyocytes” when applied to cardiomyocytes refers to the phenotype of a cell that comprises a phenotype similar to adult cardiomyocytes and does not comprise at least one feature of a fetal cardiomyocyte. In some embodiments, markers which indicate increased maturity of an in vitro-differentiated cell include, but are not limited to, electrical maturity, metabolic maturity, genetic marker maturity, and contractile maturity. The methods and compositions can comprise one or more methods for inducing cardiomyocyte maturation as described in e.g., US2020/008588 or WO 2020/190939, the contents of each of which are incorporated herein by reference in their entirety).

As used herein, the terms “transplanting,” “administering” or “engraftment” are used in the context of the placement of cells, e.g., stem cell-derived cardiomyocytes or cardiac progenitor cells, as described herein into a subject, by a method or route which results in at least partial localization of the introduced cells at a desired site, such as a site of injury or repair, such that a desired effect(s) is produced. The cells e.g., cardiac stem or progenitor cells or cardiomyocytes can be implanted directly to the heart or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of cardiomyocytes is desired as cardiomyocytes do not proliferate to an extent that the heart can heal from an acute injury comprising cell death. Thus, a graft can be used to replace lost cells that occur during injury. In other embodiments, the cells can be administered via an indirect systemic route of administration, such as an intraperitoneal or intravenous route. Methods for improving engraftment or preventing engraftment arrhythmias, such as those described in e.g., US 2020-0085880, WO 2020/190739, or WO 2021/163037, the contents of each of which are incorporated herein by reference in their entirety, can be combined with the methods and compositions described herein.

As used herein, the term “contacting” when used in reference to a cell, encompasses introducing an agent, surface, scaffold etc. to the cell in a manner that permits physical contact of the cell with the agent, surface, scaffold etc.

As used herein, the term, “cardiac disease” refers to a disease that affects the cardiac tissue of a subject. Non-limiting examples of cardiac diseases include cardiomyopathy, cardiac arrhythmias, myocardial infarction, heart failure, cardiac hypertrophy, long QT syndrome, arrhythmogenic right ventricular dysplasia (ARVD), catecholaminergic polymorphic ventricular tachycardia (CPVT), Barth syndrome, congenital defects, and Duchenne muscular dystrophy.

As used herein, the term “treatment” refers herein to the reduction of immune rejection of a cardiac graft in the presence of a combination treatment of a calcineurin inhibitor (e.g., tacrolimus) and an inhibitor of CD80 and/or CD86 signaling (e.g., abatacept) by at least 50% as compared to the amount of immune rejection in a substantially similar cardiac graft in a subject that has not been treated with the calcineurin inhibitor and an inhibitor of CD80 and/or CD86 signaling. In other embodiments, the reduction of immune rejection in a subject treated as described herein is at least 60%, at least 70%, at least 75%, at least 80%, at least 85%, at least 90%, at least 95%, at least 99% or even 100% reduction (i.e., the absence of detectable immune rejection).

As used herein, the term “immune rejection” refers to the immune-mediated death of cells transplanted at a cardiac graft site in a recipient or subject in need thereof. Immune rejection can be assessed by measuring an increase in inflammation at the graft site, for example, by an increase in circulating inflammatory markers (e.g., C-reactive protein, cytokines etc), infiltration of white blood cells at the graft site, or a need for treatment with an immunosuppressive agent or an increased dose or frequency of administration of an immunosuppressive agent. Alternatively, or in addition, immune rejection can be assessed by monitoring cardiac function or loss thereof upon immune rejection, for example, the appearance of, or increase in arrhythmias, or impairment of another cardiac function measurement.

As used herein, the term “CD3+ T cell or CD20+ B cell infiltration into the graft site is reduced” refers to at least a 20% reduction in the number of CD3+ or CD20+ B cells into the graft site in a subject treated with a calcineurin inhibitor and a CD80/CD86 signaling inhibitor as compared to the number of CD3+ or CD20+ B cells in the graft site of a subject that is not treated with such a combination. In other embodiments, the number of CD3+ or CD20+ B cells is reduced by at least 30%, at least 40%, at least 50%, at least 60%, at least 70%, at least 80%, at least 90%, at least 95%, at least 99%, or even 100% (i.e., absence of CD3+ of CD20+ B cells) when the subject is treated with a calcineurin inhibitor and a CD80/CD86 signaling inhibitor as compared to the number of CD3+ or CD20+ B cells in a subject that is not treated with such a combination.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease. A subject can be of any age including, e.g., a fetus, a neonate, a toddler, a child, an adolescent, an adult, a geriatric subject etc.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 1⁰⁰% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means +/−5%.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It will be appreciated that, although specific embodiments of the invention have been described herein for purposes of illustration, various modifications may be made without deviating from the spirit and scope of the invention. Accordingly, the invention is not limited except as by the claims.

### Cardiovascular Diseases

The methods and compositions described herein relate to methods and compositions for the extension of cardiac graft viability or prevention/reduction in cardiac graft rejection in a subject treated with a combination of a calcineurin inhibitor (e.g., tacrolimus) and an inhibitor of CD80 and/or CD86 signaling (e.g., abatacept). The methods and compositions provided herein include administering cardiomyocytes derived in vitro from pluripotent stem cells. The methods described herein can be used to treat, ameliorate, prevent or slow the progression of a number of diseases or their symptoms, such as those resulting in pathological damage to the structure and/or function of the heart.

A cardiovascular disease is a disease that affects the heart and/or circulatory system of a subject. Such cardiac diseases or cardiac-related disease include, but are not limited to, myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, arrhythmogenic right ventricular dysplasia (ARVD), long QT syndrome, catecholaminergic polymorphic ventricular tachycardia (CPVT), Barth syndrome, valvular stenosis, regurgitation, ischemia, fibrillation, polymorphic ventricular tachycardia, and muscular dystrophies such as Duchenne or related cardiac disease, and cardiomegaly.

Symptoms of cardiovascular disease can include but are not limited to syncope, fatigue, shortness of breath, chest pain, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia. Methods of diagnosing arrhythmias are known in the art.

In some embodiments of any of the aspects, the subject has or is at risk for having a cardiovascular disease or a cardiac event.

In some embodiments of any of the aspects, the subject having a cardiovascular disease is in need of, is receiving or has received a cardiac cell graft.

### Pluripotent Stem Cell Sources

The methods and compositions described herein can use cardiomyocytes differentiated in vitro, e.g., from embryonic stem cells, pluripotent stem cells, such as induced pluripotent stem cells, or other stem cells that permit such differentiation. The following describes various stem cells that can be used to prepare cardiomyocytes.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Cardiomyocytes useful in the methods and compositions described herein can be differentiated from both embryonic stem cells and induced pluripotent stem cells, among others. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells and methods for their retrieval are well known in the art and are not described in detail herein. A cell has the phenotype of an embryonic stem cell if it possesses one or more of the unique characteristics of an embryonic stem cell such that that cell can be distinguished from other cells. Exemplary distinguishing embryonic stem cell characteristics include, without limitation, morphology, gene expression or marker profile, proliferative capacity, differentiation capacity, karyotype, responsiveness to particular culture conditions, and the like.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns.

In some embodiments, the methods and compositions described herein utilize cardiomyocytes that are differentiated in vitro from induced pluripotent stem cells. An advantage of using iPSCs to generate cardiomyocyte for the compositions described herein is that the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte cell to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects. However, even cardiac grafts from autologous sources pose some risk of transplant rejection, in particular, due to the presence of neo-epitopes (e.g., mutations in mitochondrial genes so a cell can be recognized as foreign).

In some embodiments, the cardiomyocytes useful for the compositions described herein are derived from non-autologous or allogeneic sources.

In some embodiments, an iPSC is a cell that has been reprogrammed, a process that alters or reverses the differentiation state of a differentiated cell (e.g., a somatic cell). Stated another way, reprogramming is a process of driving the differentiation of a cell backwards to a more undifferentiated or more primitive type of cell. Reprogramming of somatic cells to induced pluripotent stem cells is known in the art and is not described in detail herein. iPS cells can be generated or derived from terminally differentiated somatic cells, as well as from adult stem cells, or somatic stem cells. That is, a non-pluripotent progenitor cell can be rendered pluripotent or multipotent by reprogramming.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

In vitro differentiation of Cardiomyocytes

The methods and compositions described herein can use grafts comprising in vitro-differentiated cardiomyocytes. Methods for the differentiation of cardiomyocytes from ESCs or IPSCs are known in the art. See, e.g., LaFlamme et al., Nature Biotech 25:1015-1024 (2007), which describes the differentiation of cardiomyocytes). These approaches use various factors and conditions to activate and guide differentiation programs leading to their respective cell types. Pathways and certain of the factors involved in them are discussed in the following.

In certain embodiments, the step-wise differentiation of ESCs or iPSCs to cardiomyocytes proceeds in the following order: ESC or iPSC >cardiogenic mesoderm >cardiac progenitor cells >cardiomyocytes (see e.g., US 2017024086, the contents of which are incorporated herein by reference in its entirety).

As will be appreciated by those of skill in the art, in vitro-differentiation of cardiomyocytes produces an end-result of a cell having the phenotypic and morphological features of a cardiomyocyte but that the differentiation steps of in vitro-differentiation need not be the same as the differentiation that occurs naturally in the embryo. That is, during differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis.

Exemplary methods for generating cardiomyocytes or cardiac progenitor cells from pluripotent stem cells (e.g., embryonic stem cells or induced pluripotent stem cells) are described in e.g., US2020-0085880, the contents of which are incorporated herein by reference in their entirety.

### Calcineurin Inhibitors

Calcineurin is a calcium and calmodulin dependent serine/threonine protein phosphatase (also known as protein phosphatase 3, and calcium-dependent serine-threonine phosphatase) that is ubiquitously expressed throughout the body. Calcineurin serves to activate T cells of the immune system and there are several agents that can inhibit this effect of calcineurin. Exemplary calcineurin inhibitors include small molecules, such as tacrolimus or pimecrolimus, or peptides.

In one embodiment, the calcineurin inhibitor is a small molecule. Non-limiting small molecule inhibitors of calcineurin include, but are not limited to, Cyclosporin A (CsA) and CsA derivatives (e.g., [(R)α-Methylsarcosine³]CsA, [Dimethylaminoethylthiosarcosine³]CsA, [MeBm2t]1-CsA, ISA247 (voclosporin)); FK506 (tacrolimus) and derivatives (e.g., FK520 (ascomycin), pimecrolimus (SDZ AM 981, 33-epi-chlor-33desoxy-ascomycin), L-732,531 (32-O-(1-hydroxyethylindol-5-yl)-ascomycin), L-685,818 (FK506BD) and V-10,367); FMPP (4-(fluoromethyl)phenyl phosphate), tyrphostins, norcantharidin, okadaic acid, endothall, kaempferol, barbiturates, 1,5-dibenzoyloxymethyl-norcantharidin, gossypol, Lie120, PD144795, dibenifin, dipyridamole, NCI3, INCA compounds, BTPs or 3,5-bis(trifluoromethyl)pyrazoles, BTP1, BTP2 (YM-58483), BTP3, BTP A-285222, ST1959, AM404, UR-1505, Triflusal, rocoglamide derivatives, WIN 53071, trifluoroperazine, KRM-III, caffeic acid phenyl ethyl ester (CAPE), YM-53792, quinazolinediones, pyrrolopyrimidinediones, NFAT-68, NFAT-133, punicalagin, imperatorin and quinolone alkaloids.

In one embodiment, the calcineurin inhibitor is CN585 (6-(3,4-dichlorophenyl)-4-(N,N-dimetlhylaminoethylthio)-2-phenyl-pyrimidine).

In another embodiment, the calcineurin inhibitor is a peptide. Exemplary peptides include, but are not limited to, AID fragments (derived from the autoinhibitory domain of the calcineurin) such as AID₄₂₄₋₅₂₁, AID₄₅₇₋₄₈₂, AID₄₂₀₋₅₁₁, AID₃₂₈₋₅₁₁, 11R-AID₄₅₇₋₄₈₂; PxIxIT peptides (derived from the conserved calcineurin docking motif PxIxIT found in NFATc). VIVIT 16mer oligopeptide, NFATc2₁₀₆₋₁₂₁-SPRIEIT peptide, AKAP79₃₃₀₋₃₅₇ peptide, RCANI peptide, RCANI-4₁₄₁₋₁₉₇exon7, RCANI-4₁₄₃₋₁₆₃-CIC peptide, LxVPcI peptide, RCAN1-4₉₅₋₁₁₈-SP repeat peptide, VacA peptide, A238L and A238L₂₀₀₋₂₁₃ Another example of peptides is pS3 peptide (derived from the cofilin phosphorylation domain).

In certain embodiments, the calcineurin inhibitor is FK506 (tacrolimus) or FK506 derivatives comprising calcineurin inhibitor activity, such as FK520 (ascomycin), FK523, pimecrolimus (SDZ AM 981, 33-epi-chlor-33desoxy-ascomycin), L-732,531 (32-O-(1-hydroxyethylindol-5-yl)-ascomycin), L-685,818 (FK506BD), L-732-731, 15-O-DeMe-FK-520, meridamycin, 31-O-Demethyl-FK506, V-10,367, L-683,590, L-685,818, C 18-OH-ascomycin; 9-deoxo-31-O-demethyl-FK506; L-688,617; A-1 19435 and AP1903

In one embodiment, the calcineurin inhibitor comprises tacrolimus. Without wishing to be bound by theory, the mechanism of action of tacrolimus can comprise, in part, T-lymphocyte inactivation and immunosuppression.

In some embodiments, the calcineurin inhibitor comprises a tacrolimus derivative, such as those described in U.S. Pat. No. 9,505,779, the contents of which are incorporated herein by reference in their entirety.

Inhibitors of CD80 and/or CD86 Signaling

CD80 is a transmembrane protein that provides a costimulatory signal for T cell activation. It works in tandem with CD86 to prime T cells. T regulatory cells secrete soluble CTLA-4 (sCTLA-4), which binds CD80 and blocks the co-stimulatory activation of T cells.

The methods and compositions described herein use inhibitors of CD80 and/or CD86 signaling to reduce or prevent cardiac graft rejection. CD80 and/or CD86 signaling inhibitors can include: (i) a soluble extracellular domain of CTLA4; (ii) a mutated soluble extracellular domain of CTLA4, wherein (a) an alanine at position 29 is substituted with an amino acid selected from the group consisting of tyrosine, leucine, tryptophan, and threonine, and (b) a leucine at position 104 is substituted with a glutamic acid; or (iii) the soluble extracellular domain of (i) or (ii) and a moiety which alters the solubility and/or affinity to CD80 and/or CD86 of the extracellular domain of CTLA4. In some embodiments, the moiety which alters the solubility and/or affinity to CD80 and/or CD86 of the extracellular domain of CTLA4 can be a non-proteinaceous moiety such as polyethylene glycol (PEG), or it can be a fraction of an immunoglobulin molecule, such as the Fc, constant region, of an IgG.

In one embodiment, the CD80 and/or CD86 signaling inhibitor comprises abatecept. Abatacept (marketed under the trade name ORENCIA® in both the United States and Europe) is a genetically engineered fusion protein composed of a modified Fc region of the immunoglobulin IgG1 fused to the extracellular domain of cytotoxic T-lymphocyte-associated antigen 4 (CTLA-4).

In other embodiments, the CD80 and/or CD86 signaling inhibitor comprises belatacept. Belatacept is a derivative of abatacept comprising two amino acid alterations in the CD80/86 binding portion of the abatacept compound. Belatacept comprises a 10-fold increase in the ability to inhibit T-cell activation in vitro compared to abatacept.

### Dosage and Administration

Pharmaceutical or therapeutic compositions comprising a calcineurin inhibitor and/or a CD80/CD86 signaling inhibitor for the reduction or prevention cardiac graft rejection can contain a physiologically tolerable carrier, wherein the therapeutic agent is dissolved or dispersed therein as an active ingredient(s). In a preferred embodiment, the pharmaceutical composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological or pharmaceutical composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising a therapeutic agent for reduction or prevention of rejection of a cardiac graft can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment or prevention of cardiac graft rejection depends on a number of factors, and can be determined by standard clinical techniques.

A pharmaceutical composition as described herein can be formulated for parenteral administration, e.g., by bolus injection or continuous infusion. Formulations for injection can be presented in unit dosage form, e.g., in ampoules or in multidose containers with, optionally, an added preservative. The compositions can be suspensions, solutions, or emulsions in oily or aqueous vehicles, and can contain formulatory agents such as suspending, stabilizing, and/or dispersing agents.

Pharmaceutical compositions for parenteral administration include aqueous solutions of the active preparation in water-soluble form. Additionally, suspensions of the active ingredients can be prepared as appropriate oily or water-based injection suspensions.

Suitable lipophilic solvents or vehicles include fatty oils such as sesame oil, or synthetic fatty acid esters such as ethyl oleate, triglycerides, or liposomes. Aqueous injection suspensions can contain substances that increase the viscosity of the suspension, such as sodium carboxymethyl cellulose, sorbitol, or dextran.

Optionally, the suspension can also contain suitable stabilizers or agents that increase the solubility of the active ingredients, to allow for the preparation of highly concentrated solutions. Alternatively, the active ingredient can be in powder form for constitution with a suitable vehicle. e.g., a sterile, pyrogen-free, water-based solution, before use.

In some embodiments, a therapeutic agent can be delivered in an immediate release form. In other embodiments, the therapeutic agent can be delivered in a controlled-release system or sustained-release system. Controlled- or sustained-release pharmaceutical compositions can have a common goal of improving drug therapy over the results achieved by their non-controlled or non-sustained-release counterparts. Advantages of controlled- or sustained-release compositions include extended activity of the therapeutic agents, reduced dosage frequency, and increased compliance. In addition, controlled- or sustained-release compositions can favorably affect the time of onset of action or other characteristics, such as blood levels of the therapeutic agent, and can thus reduce the occurrence of adverse side effects. Controlled- or sustained-release of an active ingredient can be stimulated by various conditions, including but not limited to, changes in pH, changes in temperature, concentration or availability of enzymes, concentration or availability of water, or other physiological conditions or compounds.

In one embodiment, a pump can be used for administration (Langer, Science 249:1527-1533 (1990); Sefton, CRC Crit. Ref Biomed. Eng. 14:201 (1987); Buchwald et al., Surgery 88:507 (1980), and Saudek et al., N. Engl J. Med 321:574 (1989)). In another embodiment, polymeric materials can be used (see Medical Applications of Controlled Release (Langer and Wise eds, 1974), Controlled Drug Bioavailability, Drug Product Design and Performance (Smolen and Ball eds., 1984); Ranger and Peppas, J. Macromol. Sci. Rev. Macromol Chem 23.61 (1983), Levy et al., Science 228:190 (1985); During et al, Ann. Neurol. 25:351 (1989); and Howard et al., J. Neurosurg. 71:105 (1989)).

When in tablet or pill form, a pharmaceutical composition as described herein can be coated (e.g., enterically coated) to delay disintegration and absorption in the gastrointestinal tract, thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

The pharmaceutical composition as described herein can also be formulated in rectal compositions such as suppositories or retention enemas, using, for example, conventional suppository bases such as cocoa butter or other glycerides.

The appropriate dosage range for a given therapeutic agent depends upon the potency, and includes amounts large enough to produce the desired effect, e.g., reduction in at least one symptom of cardiac graft rejection. The dosage of the therapeutic agent should not be so large as to cause unacceptable or life-threatening adverse side effects or should be used under close supervision by a medical professional. Generally, the dosage will vary with the type of agent, and with the age, condition, and sex of the patient. The dosage can be determined by one of skill in the art and can also be adjusted by the individual physician in the event of any complication.

Administration of the doses recited above or as employed by a skilled clinician can be repeated for a limited and defined period of time. In some embodiments, the doses are given once a day, or multiple times a day, for example, but not limited to three times a day. Typically, the dosage regimen is informed by the half-life of the agent as well as the minimum therapeutic concentration of the agent in blood, serum or localized in a given biological tissue. In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose.

A therapeutically effective amount is an amount of an agent that is sufficient to produce a statistically significant, measurable change of a given symptom of cardiac graft rejection (see “Efficacy Measurement” below). Such effective amounts can be gauged in clinical trials as well as animal studies for a given agent. For example, reduction or prevention of graft rejection can be indicative of adequate therapeutic efficacy of an agent(s).

Agents useful in the methods and compositions described herein can be administered intravenously (by bolus or continuous infusion), orally, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one therapeutic agent can be conventionally administered in a unit dose. The term “unit dose” when used in reference to a therapeutic composition refers to physically discrete units suitable as unitary dosage for the subject, each unit containing a predetermined quantity of a therapeutic agent calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

In certain embodiments, the calcineurin inhibitor is tacrolimus, which can be administered via subcutaneous, intravenous or oral administration. Exemplary therapeutic doses of tacrolimus for use with the methods and compositions described herein are administered to achieve a blood, plasma or serum concentration of 1-50 ng/mL, for example 1-45 ng/mL, 1-40 ng/mL, 1-35 ng/mL, 1-30 ng/mL, 1-25 ng/mL, 1-20 ng/mL, 1-15 ng/mL, 1-10 ng/mL, 1-9 ng/mL, 1-8 ng/mL, 1-7 ng/mL, 1-6 ng/mL, 1-5 ng/mL, 1-4 ng/mL, 1-3 ng/mL, 1-2 ng/mL, 45-50 ng/mL, 40-50 ng/mL, 35-50 ng/mL, 30-50 ng/mL, 25-50 ng/mL, 20-50 ng/mL, 15-50 ng/mL, 10-50 ng/mL, 5-50 ng/mL, 5-25 ng/mL, 5-20 ng/mL, 5-30 ng/mL, 1-25 ng/mL, 10-20 ng/mL, 10-30 ng/mL, 10-15 ng/mL, 7.5-20 ng/mL, or any integer therebetween, or an equivalent dose administered using an alternate treatment regimen. Dosages for tacrolimus derivatives, such as pimecrolimus, can be similar, adjusted up or down by the ordinarily skilled artisan to account for differences in pharmacokinetics or other parameters.

In certain embodiments, the calcineurin inhibitor comprises cyclosporin, which can be administered orally or intravenously. In embodiments where cyclosporin is administered orally, the dose of cyclosporine is within the range of 0.1-50 mg/kg daily, 0.1-40 mg/kg daily, 0.1-30 mg/kg daily, 0.1-25 mg/kg daily, 0.1-20 mg/kg daily, 0.1-15 mg/kg daily, 0.1-10 mg/kg daily, 0.1-5 mg/kg daily, 0.1-2 mg/kg daily, 40-50 mg/kg daily, 30-50 mg/kg daily, 20-50 mg/kg daily, 10-50 mg/kg daily, 5 mg/kg daily, 0.5-20 mg/kg daily, 0.5-15 mg/kg daily, 1-15 mg/kg daily, 2-15 mg/kg daily, 3-15 mg/kg daily, 4-15 mg/kg daily, 5-15 mg/kg daily, 6-15 mg/kg daily, 7-15 mg/kg daily, 8-15 mg/kg daily, 9-15 mg/kg daily, 10-15 mg/kg daily, 11-15 mg/kg daily, 12-15 mg/kg daily, 13-15 mg/kg daily, 14-15 mg/kg daily, 15-20 mg/kg daily, 16-20 mg/kg daily, 17-20 mg/kg daily, 18-20 mg/kg daily, 19-20 mg/kg daily, or any integer therebetween, or an equivalent dose administered using an alternate treatment regimen. In one embodiment, the dose of cyclosporin administered orally comprises 1-15 mg/kg daily.

In embodiments where cyclosporin is administered intravenously, the dose of cyclosporine is within the range of 0.1-15 mg/kg daily, 0.1-14 mg/kg daily, 0.1-13 mg/kg daily, 0.1-12 mg/kg daily, 0.1-10 mg/kg daily, 0.1-8 mg/kg daily, 0.1-5 mg/kg daily, 0.1-4 mg/kg daily, 0.1-3 mg/kg daily, 0.1-2 mg/kg daily, 0.1-1 mg/kg daily, 1-15 mg/kg daily, 1-14 mg/kg daily, 1-13 mg/kg daily, 1-12 mg/kg daily, 1-11 mg/kg daily, 1-10 mg/kg daily, 1-9 mg/kg daily. 1-8 mg/kg daily, 1-7 mg/kg daily, 1-6 mg/kg daily, 1-5 mg/kg daily, 1-4 mg/kg daily, 1-3 mg/kg daily, 1-2 mg/kg daily, 2-15 mg/kg daily, 2-14 mg/kg daily, 2-13 mg/kg daily, 2-12 mg/kg daily, 2-11 mg/kg daily, 2-10 mg/kg daily, 2-9 mg/kg daily, 2-8 mg/kg daily, 2-7 mg/kg daily, 2-6 mg/kg daily, 2-5 mg/kg daily, 2-4 mg/kg daily, 2-3 mg/kg daily, 3-15 mg/kg daily, 3-14 mg/kg daily, 3-13 mg/kg daily, 3-12 mg/kg daily, 3-11 mg/kg daily, 3-10 mg/kg daily, 3-9 mg/kg daily, 3-8 mg/kg daily, 3-7 mg/kg daily, 3-6 mg/kg daily, 3-5 mg/kg daily, 3-4 mg/kg daily, 4-15 mg/kg daily, 4-14 mg/kg daily, 4-13 mg/kg daily, 4-12 mg/kg daily, 4-11 mg/kg daily, 4-10 mg/kg daily, 4-9 mg/kg daily, 4-8 mg/kg daily, 4-7 mg/kg daily, 4-6 mg/kg daily, 4-5 mg/kg daily, 5-15 mg/kg daily, 5-14 mg/kg daily, 5-13 mg/kg daily, 5-12 mg/kg daily, 5-11 mg/kg daily, 5-10 mg/kg daily. 5-9 mg/kg daily, 5-8 mg/kg daily, 5-7 mg/kg daily, 5-6 mg/kg daily, 6-15 mg/kg daily, 6-14 mg/kg daily, 6-13 mg/kg daily, 6-12 mg/kg daily, 6-11 mg/kg daily, 6-10 mg/kg daily, 6-9 mg/kg daily, 6-8 mg/kg daily, 6-7 mg/kg daily, 7-15 mg/kg daily, 8-15 mg/kg daily, 9-15 mg/kg daily, 10-15 mg/kg daily, Il-15 mg/kg daily, 12-15 mg/kg daily, 13-15 mg/kg daily, 14-15 mg/kg daily, or any integer therebetween, or an equivalent dose administered using an alternate treatment regimen. In one embodiment, the dose of cyclosporin administered intravenously comprises 1-6 mg/kg daily.

In certain embodiments, the calcineurin inhibitor comprises voclosporin, which can be administered orally twice daily with a dose in the range of 0.01-5 mg/kg, 0.01-4 mg/kg, 0.01-3 mg/kg, 0.01-2 mg/kg, 0.01-1 mg/kg, 0.1-5 mg/kg, 0.1-4 mg/kg, 0.1-3 mg/kg, 0.1-2 mg/kg daily, 0.1-1 mg/kg, 0.1-0.5 mg/kg, 0.5-5 mg/kg, 0.5-4 mg/kg, 0.5-3 mg/kg, 0.5-2 mg/kg, 0.5-1 mg/kg, 1-5 mg/kg, 1-4 mg/kg, 1-3 mg/kg, 1-2 mg/kg, 2-5 mg/kg, 2-4 mg/kg, 2-3 mg/kg, 3-5 mg/kg, 3-4 mg/kg, 4-5 mg/kg or any integer therebetween, or an equivalent dose administered using a different treatment regimen. In one embodiment, the dose of voclosporin administered twice a day via oral administration comprises 0.1-1 mg/kg.

In certain embodiments, the inhibitor CD80 and/or CD86 signaling comprises abatacept or belatacept, which can be administered either intravenously or subcutaneously. In some embodiments, the dose of abatacept or belatacept is 1.25-125 mg/kg administered intravenously or subcutaneously at a given interval (e.g., every 10 days, every 11 days, every 12 days, every 13 days, every 14 days, every 15 days, every 16 days, every 17 days, every 18 days, every 19 days, ever 20 days, every 21 days, every 22 days, every 23 days, every 3 weeks, every 4 weeks or more). In certain embodiments, the intravenous or subcutaneous dose of abatacept or belatacept is 1.25-100 mg/kg, 1.25-90 mg/kg, 1.25-80 mg/kg, 1.25-75 mg/kg, 1.25-70 mg/kg, 1.25-60 mg/kg, 1.25-50 mg/kg, 1.25-40 mg/kg, 1.25-30 mg/kg, 1.25-20 mg/kg, 1.25-10 mg/kg, 1.25-5 mg/kg, 2-125 mg/kg, 5-125 mg/kg, 10-125 mg/kg, 10-125 mg/kg, 20-125 mg/kg, 30-125 mg/kg, 40-125 mg/kg, 50-125 mg/kg, 60-125 mg/kg, 70-125 mg/kg, 75-125 mg/kg, 80-125 mg/kg. 90-125 mg/kg, 100-125 mg/kg, 110-125 mg/kg, 115-125 mg/kg, 5-20 mg/kg, 5-15 mg/kg, 5-25 mg/kg, 10-20 mg/kg, 10-25 mg/kg, 20-50 mg/kg, 25-75 mg/kg or any integer therebetween, or an equivalent dose administered using an alternate treatment regimen.

In certain embodiments, the dose of abatacept or belatacept is 5-25 mg/kg, and the therapeutic dose of tacrolimus is administered to achieve a blood, plasma or serum concentration is selected from: 1-50 ng/mL, for example 1-45 ng/mL, 1-40 ng/mL, 1-35 ng/mL, 1-30 ng/mL, 1-25 ng/mL, 1-20 ng/mL, 1-15 ng/mL, 1-10 ng/mL, 1-9 ng/mL, 1-8 ng/mL, 1-7 ng/mL, 1-6 ng/mL, 1-5 ng/mL, 1-4 ng/mL, 1-3 ng/mL, 1-2 ng/mL, 45-50 ng/mL, 40-50 ng/mL, 35-50 ng/mL, 30-50 ng/mL, 25-50 ng/mL, 20-50 ng/mL, 15-50 ng/mL, 10-50 ng/mL, 5-50 ng/mL, 5-25 ng/mL, 5-20 ng/mL, 5-30 ng/mL, 1-25 ng/mL, 10-20 ng/mL, 10-30 ng/mL, 10-15 ng/mL, 7.5-20 ng/mL, or any integer therebetween, or an equivalent dose administered using an alternate treatment regimen.

In certain embodiments, a dose of tacrolimus is administered to achieve a blood, plasma or serum concentration of 5-30 ng/mL in combination with a dose of abatacept or belatacept selected from the group consisting of: 1.25-100 mg/kg, 1.25-90 mg/kg, 1.25-80 mg/kg, 1.25-75 mg/kg, 1.25-70 mg/kg, 1.25-60 mg/kg, 1.25-50 mg/kg, 1.25-40 mg/kg, 1.25-30 mg/kg, 1.25-20 mg/kg, 1.25-10 mg/kg, 1.25-5 mg/kg, 2-125 mg/kg, 5-125 mg/kg, 10-125 mg/kg, 10-125 mg/kg, 20-125 mg/kg, 30-125 mg/kg, 40-125 mg/kg, 50-125 mg/kg, 60-125 mg/kg, 70-125 mg/kg, 75-125 mg/kg, 80-125 mg/kg, 90-125 mg/kg, 100-125 mg/kg, 110-125 mg/kg, 115-125 mg/kg, 5-20 mg/kg, 5-15 mg/kg, 5-25 mg/kg, 10-20 mg/kg, 10-25 mg/kg, 20-50 mg/kg, 25-75 mg/kg or any integer therebetween, or an equivalent dose administered using an alternate treatment regimen.

In certain embodiments, the dose of tacrolimus is administered to achieve a blood, plasma or serum concentration of 5-25 ng/mL and the dose of abatacept or belatacept is 5-20 mg/kg.

In some embodiments of the combination treatment, the calcineurin inhibitor is selected from the group consisting of: tacrolimus, cyclosporine, voclosporin, and pimecrolimus. In some embodiments of the combination treatment, the inhibitor of CD80 and/or CD86 comprises an extracellular domain fragment of CTLA4 In some embodiments of the combination treatment, the inhibitor of CD80 and/or CD86 signaling comprises abatacept or belatacept. In some embodiments, the combination treatment comprises the calcineurin inhibitor tacrolimus and the inhibitor of CD80 and/or CD86 signaling, abatacept. In some embodiments, the combination treatment comprises the calcineurin inhibitor cyclosporine and the inhibitor of CD80 and/or CD86 signaling, abatacept. In some embodiments, the combination treatment comprises the calcineurin inhibitor voclosporin and the inhibitor of CD80 and/or CD86 signaling, abatacept. In some embodiments, the combination treatment comprises the calcineurin inhibitor pimecrolimus and the inhibitor of CD80 and/or CD86 signaling, abatacept. In some embodiments, the combination treatment comprises the calcineurin inhibitor tacrolimus and the inhibitor of CD80 and/or CD86 signaling, belatacept. In some embodiments, the combination treatment comprises the calcineurin inhibitor cyclosporine and the inhibitor of CD80 and/or CD86 signaling, belatacept. In some embodiments, the combination treatment comprises the calcineurin inhibitor voclosporin and the inhibitor of CD80 and/or CD86 signaling, belatacept. In some embodiments, the combination treatment comprises the calcineurin inhibitor pimecrolimus and the inhibitor of CD80 and/or CD86 signaling, belatacept. In some embodiments, the combination treatment comprises the calcineurin inhibitor tacrolimus and the inhibitor of CD80 and/or CD86 signaling comprises an extracellular domain fragment of CTLA4. In some embodiments, the combination treatment comprises the calcineurin inhibitor cyclosporine and the inhibitor of CD80 and/or CD86 signaling, comprises an extracellular domain fragment of CTLA4. In some embodiments, the combination treatment comprises the calcineurin inhibitor voclosporin and the inhibitor of CD80 and/or CD86 signaling comprises an extracellular domain fragment of CTLA4. In some embodiments, the combination treatment comprises the calcineurin inhibitor pimecrolimus and the inhibitor of CD80 and/or CD86 signaling comprises an extracellular domain fragment of CTLA4.

In some embodiments, it is contemplated that the combination therapy will persist for at least the lifetime of the graft and up to the remaining lifetime of the recipient in need thereof. The immune system is dynamic and can flare in response to viral or bacterial infection, which can then initiate an autoimmune rejection against the graft, thus long-term treatment as described herein is contemplated.

### Combination Therapies

Treatment of a subject with both a calcineurin inhibitor and an inhibitor of CD80 and/or CD86 signaling is considered a “combination therapy.” In other embodiments, the therapeutically effective agents (i.e., a calcineurin inhibitor and a CD80/CD86 signaling inhibitor) described herein are administered to a subject concurrently with an additional combination therapy, such as an immunosuppressive agent. Exemplary immunosuppressive agents can include, but are not limited to, methylprednisolone, betamethasone, cortisone, dexamethasone, hydrocortisone, prednisolone, prednisone, or triamcinolone. An exemplary treatment regimen for cardiac graft rejection comprises 500 mg methylprednisolone administered IV until separation from cardiopulmonary bypass, followed by 125 mg methylprednisolone administered IV every 8 hours for the first 24 hours and then the subject is switched to oral prednisone at a dose of 0.1-10 mg/kg PO daily.

As used herein, the term “concurrently” is not limited to the administration of the two or more agents at exactly the same time, but rather, it is meant that they are administered to a subject in a sequence and within a time interval such that they can act together (e.g., synergistically to provide an increased benefit than if they were administered alone). For example, the combination of therapeutics can be administered at the same time or sequentially in any order at different points in time; however, if not administered at the same time, they should be administered sufficiently close in time so as to provide the desired therapeutic effect, preferably in a synergistic fashion. The agents can be administered separately, in any appropriate form and by any suitable route. When each of the therapeutic agents in a combination are not administered in the same pharmaceutical composition, it is understood that they can be administered in any order to a subject in need thereof. For example, the first therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second therapeutic agent, to a subject in need thereof (or vice versa). In other embodiments, the delivery of either therapeutic agent ends before the delivery of the other agent/treatment begins. In some embodiments of either case, the treatment is more effective because of combined administration. For example, the therapeutic agents used in combination are more effective than would be seen with either agent alone. In some embodiments, delivery is such that the reduction in a symptom, or other parameter related to the disorder is greater than what would be observed with either therapeutic agent alone. The effect of such a combination can be partially additive, wholly additive, or greater than additive. The agent and/or other therapeutic agents, procedures or modalities can be administered during periods of active disease, or during a period of persistence or less active disease.

When administered in combination, one or more of the therapeutic agents can be administered in an amount or dose that is higher, lower or the same as the amount or dosage of the given agent used individually, e.g., as a monotherapy. In certain embodiments, the administered amount or dosage of a first therapeutic agent when administered in combination with a second therapeutic agent is lower (e.g., at least 20%, at least 30%, at least 40%, or at least 50%) than the amount or dosage of the first agent when used individually. In other embodiments, the amount or dosage of a first therapeutic agent, when administered in combination with a second therapeutic agent, that results in a desired effect (e.g., improved cognitive functioning) is lower (e.g., at least 20%, at least 30%, at least 40%, or at least 50% lower) than the amount or dosage of the first (or second) agent required to achieve the same therapeutic effect when administered alone

### Efficacy

The efficacy of a given treatment for reducing or preventing cardiac graft rejection can be determined by the skilled clinician. However, a treatment is considered “effective treatment,” as the term is used herein, if any one or all of the signs or symptoms of cardiac graft rejection is/are altered in a beneficial manner, or other clinically accepted symptoms or markers of disease are improved, or ameliorated, e.g., by at least 10% following treatment with a therapeutic agent for cardiac graft rejection. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the graft rejection or (2) relieving the disease, e.g., causing regression of symptoms; and (3) preventing or reducing the likelihood of the development of the disease, or preventing secondary diseases/disorders associated with the graft rejection (e.g., cardiomyopathy etc).

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., reduction or cessation in arrhythmias, reduction in inflammatory markers (e.g., C-reactive protein (CRP)), etc.

In some embodiments, efficacy of a given combination treatment (e.g., tacrolimus and abatacept) can be determined by a reduction in severity of cellular rejection on an endomyocardial biopsy according to the International Society for Heart and Lung Transplantation (ISHLT) Acute Cellular Rejection Grading Scheme. The grading scheme comprises 4 different levels of rejection: (i) 0R, no rejection as evidenced by no histopathological findings, (ii) 1R, mild, interstitial and/or perivascular infiltrate with up to 1 focus of myocyte damage, (iii) 2R, moderate, two or more foci infiltrate with associated myocardial damage, and (iv) 3R, severe, diffuse infiltrate with multifocal myocyte damage±edema±hemorrhage±vasculitis. Thus, a given combination treatment is efficacious if the subject moves down at least one level on the ISHLT Acute Cellular Rejection Grading Scheme (e.g., from severe to moderate, from moderate to mild, from mild to none, from severe to mild, from severe to none, from moderate to none etc.). Ideally, treatment will be optimized to achieve a grading of 2R, 1R or 0R.

In some embodiments, efficacy can be measured by a reduction in the need for co-immunosuppressive therapies (e.g., glucocorticoids) without an increase in graft rejection. The reduction in need of an immunosuppressive therapy/therapies can include, for example, a reduction in dose of a given agent, removal of a co-immunosuppressive therapy from the treatment regimen, a reduction in the frequency of dosing, a reduction in the duration of therapy, or replacement of a given immunosuppressive therapy with a similar agent of less potency.

In other embodiments, efficacy can be assessed by measuring an extension of the graft viability in subjects treated with a calcineurin inhibitor and CD80/CD86 signaling inhibitor as compared to the length of graft viability in subjects that are not treated with these agents. For example, the viability of the cardiac graft is extended by at least 3 weeks, at least 4 weeks, at least 5 weeks, at least 6 weeks, at least 7 weeks, at least 8 weeks, at least 3 months, at least 4 months, at least 5 months, at least 6 months, at least 9 months, at least 1 year, at least 2 years, at least 3 years, at least 5 years, at least 10 years, at least 20 years or more when the subject is treated with a combination of a calcineurin inhibitor and an inhibitor of CD80 and/or CD86 signaling than the viability of a substantially similar cardiac graft where the subject is not treated with such a combination.

In other embodiments, efficacy can also be expressed as a reduction or elimination of cell loss in a cardiac graft when the subject is treated with a combination of a calcineurin inhibitor and an inhibitor of CD80 and/or CD86 signaling as compared to the loss that occurs in an untreated subject having a similar cardiac graft. For example, a reduction in cell loss in a cardiac graft can be less than 0.5% when the subject is treated with a calcineurin inhibitor and an inhibitor of CD80 and/or CD86 signaling compared to the graft upon initial engraftment (e.g., graft size at 1 week, 2 weeks, 3 weeks, 4 weeks, 5 weeks, 6 weeks, 7 weeks or 8 weeks); in other embodiments, treatment can be less than 1%, less than 2%, less than 5%, less than 10%, less than 20%, less than 30%, less than 40%, less than 50%, or less than 60% cell loss of the cardiac graft when the subject is treated with a calcineurin inhibitor and an inhibitor of CD80 and/or CD86 signaling.

### EXAMPLES

### Example 1: Steroid-Sparing Chronic Immunosuppression for Allogeneic Stem-Cell Derived Cardiomyocyte Transplantation

Pluripotent stem cell-derived cardiomyocyte (PSC-CM) transplantation is an emerging strategy to treat recent myocardial infarction (MI). Most studies to date have used xenotransplantation of human PSC-CM, which does not inform on how to achieve successful allotransplantation in patients. To identify a minimal, steroid-sparing immunosuppression regimen for first-in-human allogeneic transplantation, a non-human primate (NHP) model was developed where rhesus iPSC-CM are transplanted into normal or infarcted hearts of MHC-mismatched recipients. Allogeneic iPSCs derived from rhesus macaque (Macaca mulatta) were differentiated into a highly enriched population of cardiomyocytes (riPSC-CMs). 200×10⁶ riPSC-CMs were transplanted into 11 MHC-mismatched rhesus recipients. The primary study endpoint was the degree of histopathological immune rejection. In the absence of immunosuppression, cardiomyocyte grafts survived for 2 weeks without rejection, but by 8 weeks they underwent severe cellular rejection with extensive inflammatory infiltration by CD3+ T cells and CD20+ B cells leading to graft destruction. Subsequent studies focused on 8-week engraftment, with promising regimens studied further out to 16 weeks. Low-dose subcutaneous tacrolimus (TAC) alone (trough 5-10 ng/mL) or subcutaneous Abatacept (ABT, CTLA4-Ig) alone (12.5 mg/kg biweekly) were insufficient, with grafts exhibiting moderate to severe rejection at 8 weeks post-transplant. In contrast, the combination of medium-dose TAC (trough 10-15 ng/mL) and ABT (12.5 mg/kg biweekly) resulted in graft preservation with no significant myocardial rejection at 8 weeks post-transplantation. Evaluation of TAC plus ABT at 16 weeks post-transplantation, however, revealed low-moderate immune rejection of the graft with patchy infiltration of T cells and B cells in one animal and no significant rejection in a second NHP. The presence or absence of MI did not affect the outcome of immunoreactivity. In summary, allogeneic iPSC-CM are less immunogenic than whole-heart allografts, and data to date suggest that a steroid-sparing immunosuppression regimen may be possible to support long-term PSC-CM transplantation. Therefore, combinatorial immunosuppression regimen of TAC and ABT can be used to support long-term PSC-CM transplantation. This study also suggests that extended observation out to 16 weeks is required to detect late-onset rejection.

All headings and section designations are used for clarity and reference purposes only and are not to be considered limiting in any way. For example, those of skill in the art will appreciate the usefulness of combining various embodiments from different headings and sections as appropriate according to the spirit and scope of the technology described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method of cardiomyocyte transplantation, the method comprising:
administering in vitro-differentiated cardiomyocytes or cardiac progenitor cells to a graft site in cardiac tissue of a recipient in need thereof, and
administering a calcineurin inhibitor and an inhibitor of CD80 and/or CD86 signaling to the recipient.

2. The method of claim 1, wherein the in vitro-differentiated cardiomyocytes are allogeneic to the recipient.

3. The method of claim 1, wherein the in vitro-differentiated cardiomyocytes are autologous to the recipient.

4. The method of claim 1, 2, or 3 wherein the calcineurin inhibitor is selected from the group consisting of: tacrolimus, cyclosporine, voclosporin, and pimecrolimus.

5. The method of any one of claims 1-4, wherein the inhibitor of CD80 and/or CD86 comprises an extracellular domain fragment of CTLA4.

6. The method of claim 5, wherein the extracellular domain fragment of CTLA4 is fused to a heterologous polypeptide.

7. The method of claim 6, wherein the heterologous polypeptide increases the serum half-life of the inhibitor of CD80 and/or CD86.

8. The method of claim 6 or claim 7, wherein the heterologous polypeptide comprises an IgG Fc domain polypeptide.

9. The method of claim 5, wherein the extracellular domain fragment of CTLA4 is conjugated with polyethylene glycol (PEG).

10. The method of any one of claims 1-9, wherein the inhibitor of CD80 and/or CD86 signaling comprises abatacept or belatacept.

11. The method of claim 10, wherein abatacept is administered at a dose between 5 mg/kg and 20 mg/kg via intravenous or subcutaneous administration.

12. The method of any one of claims 1-11, wherein the inhibitor of CD80 and/or CD86 signaling is (i) administered every 2 weeks or every 3 weeks,
(ii) administered every 12-16 days,
(iii) administered every 10-18 days, or
(iv) administered by subcutaneous, oral or intravenous administration.

13. The method of any one of claims 1-12, wherein the calcineurin inhibitor comprises tacrolimus or an immunosuppressive derivative thereof.

14. The method of claim 13, wherein tacrolimus is administered to achieve a serum or plasma concentration of 5-20 ng/ml.

15. The method of claim 13 or 14, wherein the tacrolimus is administered via a continuous infusion, every 2 hours, every 4 hours, every 6 hours, every 8 hours, every 10 hours, every 12 hours, ever 14 hours, every 16 hours, every 18 hours, every 20 hours, every 22 hours, or daily.

16. The method of any one of claims 1-15 wherein the calcineurin inhibitor and the inhibitor of CD80 and/or CD86 signaling are each administered subcutaneously or intravenously or a combination thereof.

17. The method of any one of claims 1-16, wherein immune rejection of the administered cardiomyocytes is reduced relative to rejection of cardiomyocytes administered without the calcineurin inhibitor and the inhibitor of CD80 and/or CD86 signaling.

18. The method of any one of claims 1-17, wherein the need for steroid immunosuppression to suppress immune rejection of the transplanted cardiomyocytes is reduced relative to the need for steroid immunosuppression when cardiomyocytes are transplanted without administering the calcineurin inhibitor and the inhibitor of CD80 and/or CD86 signaling.

19. The method of any one of claims 1-18, wherein CD3+ T cell or CD20+ B cell infiltration into the graft site is reduced relative to infiltration occurring in the absence of administering the calcineurin inhibitor and the inhibitor of CD80 and/or CD86 signaling.

20. The method of claim 19, wherein CD3+ T cell or CD20+ B cell infiltration into the graft site is reduced at 8 weeks post-administration of the cardiomyocytes.

21. The method of claim 19, wherein CD3+ T cell or CD20+ B cell infiltration into the graft site remains reduced at 16 weeks post-administration of the cardiomyocytes.

22. A method of reducing immune rejection of transplanted, in vitro-differentiated cardiomyocytes in a recipient, the method comprising administering a calcineurin inhibitor and an inhibitor of CD80 and/or CD86 signaling to the recipient.

23. The method of claim 22, wherein the in vitro-differentiated cardiomyocytes are allogeneic to the recipient.

24. The method of claim 22, wherein the in vitro-differentiated cardiomyocytes are autologous to the recipient.

25. The method of claim 22, 23, or 24 wherein the calcineurin inhibitor is selected from the group consisting of: tacrolimus, cyclosporine, voclosporin, and pimecrolimus.

26. The method of any one of claims 22-25, wherein the inhibitor of CD80 and/or CD86 comprises an extracellular domain fragment of CTLA4.

27. The method of claim 26, wherein the extracellular domain fragment of CTLA4 is fused to a heterologous polypeptide.

28. The method of claim 27, wherein the heterologous polypeptide increases the serum half-life of the inhibitor of CD80 and/or CD86.

29. The method of claim 27 or claim 28, wherein the heterologous polypeptide comprises an IgG Fc domain polypeptide.

30. The method of claim 26, wherein the extracellular domain fragment of CTLA4 is conjugated with polyethylene glycol (PEG).

31. The method of any one of claims 22-30, wherein the inhibitor of CD80 and/or CD86 signaling comprises abatacept or belatacept.

32. The method of claim 31, wherein abatacept is administered at a dose between 5 mg/kg and 20 mg/kg via intravenous or subcutaneous administration.

33. The method of any one of claims 22-32, wherein the inhibitor of CD80 and/or CD86 signaling is (i) administered every 2 weeks or every 3 weeks,
(ii) administered every 12-16 days,
(iii) administered every 10-18 days, and/or
(iv) administered by subcutaneous, oral or intravenous administration.

34. The method of any one of claims 22-33, wherein the calcineurin inhibitor comprises tacrolimus or an immunosuppressive derivative thereof.

35. The method of claim 34, wherein tacrolimus is administered to achieve a serum or plasma concentration of 5-20 ng/ml.

36. The method of claim 34 or 35, wherein the tacrolimus is administered via a continuous infusion, every 2 hours, every 4 hours, every 6 hours, every 8 hours, every 10 hours, every 12 hours, ever 14 hours, every 16 hours, every 18 hours, every 20 hours, every 22 hours, or daily.

37. The method of any one of claims 22-36 wherein the calcineurin inhibitor and the inhibitor of CD80 and/or CD86 signaling are each administered subcutaneously or intravenously or a combination thereof.

38. The method of any one of claims 22-37, wherein immune rejection of the administered cardiomyocytes is reduced relative to rejection of cardiomyocytes administered without the calcineurin inhibitor and the inhibitor of CD80 and/or CD86 signaling.

39. The method of any one of claims 22-38, wherein the need for steroid immunosuppression to suppress immune rejection of the transplanted cardiomyocytes is reduced relative to the need for steroid immunosuppression when cardiomyocytes are transplanted without administering the calcineurin inhibitor and the inhibitor of CD80 and/or CD86 signaling.

40. The method of any one of claims 22-39, wherein CD3+ T cell or CD20+ B cell infiltration into the graft site is reduced relative to infiltration occurring in the absence of administering the calcineurin inhibitor and the inhibitor of CD80 and/or CD86 signaling.

41. The method of claim 40, wherein CD3+ T cell or CD20+ B cell infiltration into the graft site is reduced at 8 weeks post-administration of the cardiomyocytes.

42. The method of claim 40, wherein CD3+ T cell or CD20+ B cell infiltration into the graft site remains reduced at 16 weeks post-administration of the cardiomyocytes.

43. A method of treating a cardiovascular disease or disorder, the method comprising:
administering in vitro-differentiated cardiomyocytes to a graft site in cardiac tissue of a subject in need thereof, and
administering a calcineurin inhibitor and an inhibitor of CD80 and/or CD86 signaling to the subject.

44. The method of claim 43, wherein the cardiovascular disease or disorder comprises tachyarrhythmias, bradyarrhhythmias, cardiomyopathy, congenital defects, and muscular dystrophy-associated cardiomyopathy.

45. The method of claim 44, wherein the congenital defects comprise septal defects or hypoplastic syndroms.

46. The method of claim 44, wherein the muscular-dystrophy associated cardiomyopathy comprises cardiomyopathy associated with Duchenne's muscular dystrophy (DMD).

47. The method of claim 43, wherein the in vitro-differentiated cardiomyocytes are allogeneic to the recipient.

48. The method of claim 43, wherein the in vitro-differentiated cardiomyocytes are autologous to the recipient.

49. The method of claim 43, 44, or 45 wherein the calcineurin inhibitor is selected from the group consisting of: tacrolimus, cyclosporine, voclosporin, and pimecrolimus.

50. The method of any one of claims 43-48, wherein the inhibitor of CD80 and/or CD86 comprises an extracellular domain fragment of CTLA4.

51. The method of claim 49, wherein the extracellular domain fragment of CTLA4 is fused to a heterologous polypeptide.

52. The method of claim 50, wherein the heterologous polypeptide increases the serum half-life of the inhibitor of CD80 and/or CD86.

53. The method of claim 51 or claim 52, wherein the heterologous polypeptide comprises an IgG Fc domain polypeptide.

54. The method of claim 50, wherein the extracellular domain fragment of CTLA4 is conjugated with polyethylene glycol (PEG).

55. The method of any one of claims 43-54, wherein the inhibitor of CD80 and/or CD86 signaling comprises abatacept or belatacept.

56. The method of claim 55, wherein abatacept is administered at a dose between 5 mg/kg and 20 mg/kg via intravenous or subcutaneous administration.

57. The method of any one of claims 43-56, wherein the inhibitor of CD80 and/or CD86 signaling is (i) administered every 2 weeks or every 3 weeks,
(ii) administered every 12-16 days,
(iii) administered every 10-18 days, and/or
(iv) administered by subcutaneous, oral or intravenous administration.

58. The method of any one of claims 43-57, wherein the calcineurin inhibitor comprises tacrolimus or an immunosuppressive derivative thereof.

59. The method of claim 58, wherein tacrolimus is administered to achieve a serum or plasma concentration of 5-20 ng/ml.

60. The method of claim 58 or 59, wherein the tacrolimus is administered via a continuous infusion, every 2 hours, every 4 hours, every 6 hours, every 8 hours, every 10 hours, every 12 hours, ever 14 hours, every 16 hours, every 18 hours, every 20 hours, every 22 hours, or daily.

61. The method of any one of claims 43-60 wherein the calcineurin inhibitor and the inhibitor of CD80 and/or CD86 signaling are each administered subcutaneously or intravenously or a combination thereof.

62. The method of any one of claims 43-61, wherein immune rejection of the administered cardiomyocytes is reduced relative to rejection of cardiomyocytes administered without the calcineurin inhibitor and the inhibitor of CD80 and/or CD86 signaling.

63. The method of any one of claims 43-62, wherein the need for steroid immunosuppression to suppress immune rejection of the transplanted cardiomyocytes is reduced relative to the need for steroid immunosuppression when cardiomyocytes are transplanted without administering the calcineurin inhibitor and the inhibitor of CD80 and/or CD86 signaling.

64. The method of any one of claims 43-63, wherein CD3+ T cell or CD20+ B cell infiltration into the graft site is reduced relative to infiltration occurring in the absence of administering the calcineurin inhibitor and the inhibitor of CD80 and/or CD86 signaling.

65. The method of claim 64, wherein CD3+ T cell or CD20+ B cell infiltration into the graft site is reduced at 8 weeks post-administration of the cardiomyocytes.

66. The method of claim 65, wherein CD3+ T cell or CD20+ B cell infiltration into the graft site remains reduced at 16 weeks post-administration of the cardiomyocytes.

67. Use of a calcineurin inhibitor in combination with an inhibitor of CD80 and/or CD86 signaling for the treatment or prevention of immune-mediated rejection of a cardiac graft.

68. The use according to claim 67, comprising any of the features of claims 22-42.

69. Use of in vitro-differentiated cardiomyocytes or cardiac progenitor cells for cardiomyocyte transplantation, the use comprising:
in vitro-differentiated cardiomyocytes or cardiac progenitor cells to be administered to a graft site in cardiac tissue of a recipient in need thereof, and
a calcineurin inhibitor and an inhibitor of CD80 and/or CD86 signaling that is to be administered to the recipient.

70. The use according to claim 69, comprising any of the features of claims 2-42.

71. Use of in vitro-differentiated cardiomyocytes for treating a cardiovascular disease or disorder, the use comprising:
in vitro-differentiated cardiomyocytes to a graft site in cardiac tissue to be administered to a subject in need thereof, and
a calcineurin inhibitor and an inhibitor of CD80 and/or CD86 signaling to be administered to the subject

72. The use according to claim 71, comprising any of the features of claims 44-66.
